(19) United States
(12) Patent Application Publication
(10) Pub. No.: US 2013/0143948 A1
(43) Pub. Date: Jun. 6, 2013

(54) MST1 AS A PROGNOSTIC BIOMARKER AND THERAPEUTIC TARGET IN HUMAN CANCER

(71) Applicants: Bekir Cinar, Los Angeles, CA (US); Filiz Kisaayak Collak, Los Angeles, CA (US)

(72) Inventors: Bekir Cinar, Los Angeles, CA (US); Filiz Kisaayak Collak, Los Angeles, CA (US)

(73) Assignee: CEDARS-SINAI MEDICAL CENTER, Los Angeles, CA (US)

(21) Appl. No.: 13/816,917

(22) PCT Filed: Aug. 19, 2011
(86) PCT No.: PCT/US11/48504

§ 371 Date: Feb. 13, 2013

Related U.S. Application Data
(60) Provisional application No. 61/375,472, filed on Aug. 20, 2010.

(51) Int. Cl. G01N 33/68; A61N 5/00

(52) U.S. Cl. CPC G01N 33/6893; A61N 5/00
USPC 514/44 A; 435/7.9; 600/1

(57) ABSTRACT

The present invention relates to MST1 and MST2 cancer biomarkers. The inventors demonstrate herein that MST1 and/or MST2 can be used as biomarkers for the detection and prognosis of prostate cancer. The invention further discloses that enforced expression of MST1 can be used to inhibit and/or suppress the progression of prostate cancer.

### CROSS-REFERENCE TO RELATED APPLICATIONS

This application claims priority from U.S. Provisional Patent Application No. 61/375,472, filed on Aug. 20, 2010, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant Nos. RO1 CA124706 (DG), R01 CA143777, R01 CA112303 (MRF), W81XWH-08-1-0150 (MRF).

### FIELD OF THE INVENTION

This invention generally relates to cancer diagnosis, prognosis and treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The serine-threonine kinase MST1 or STK4 (mammalian sterile STE20-like kinase 1), a homolog of Hippo (Hpo/hpo) in Drosophila, was originally identified as a pro-apoptotic protein (1). MST1 is related to three paralogs (MST2, MST3, and MST4) with a conserved structure consisting of N-terminal catalytic (MST1-N) and C-terminal regulatory (MST1-C) domains and other functional sites, including caspase cleavage sites and nuclear export signals (2, 3). MST1 or MST2 can be activated by autophosphorylation of a unique threonine residue (Thr-183 in MST1 and Thr-180 in MST2) in the activation loop or by caspase-3 cleavage in response to a wide range of cell death stimuli (4).

In addition to their pro-apoptotic function, MST1 and its closest paralog MST2 have been demonstrated to play an important role in mammalian development (5, 6), cell cycle progression and tumorigenesis (7-10). For example, hpo deficiency in the developing Drosophila eye results in massive overgrowth due to an accelerated rate of proliferation and failure of developmental apoptosis (11-13). Likewise, MST1 or MST2 deficiency in mice is embryonically lethal (5). Loss or reduction of MST1 and MST2 expression has also been correlated with poor cancer prognosis (14). Recent genetic studies have indicated that liver-specific deletion of MST1 and MST2 in mice resulted in liver enlargement, cancer and resistance to TNF-α induced apoptosis (7, 9, 10). Previous studies suggest that cross talk between androgen receptor (AR) and MST1 signaling may have important biological consequences in prostate cancer (PCa) (15, 16).

There is a need in the art for a novel prognostic and diagnostic biomarker and therapeutic target in human cancer.

### SUMMARY OF THE INVENTION

In various embodiments, the invention teaches a method of detecting cancer in a subject including: obtaining a sample biopsy from a subject; determining the expression level of MST1 and/or MST2 in the sample biopsy; and comparing the expression level of MST1 and/or MST2 with the expression level of MST1 and/or MST2 from a control biopsy; wherein a lower level of expression of MST1 and/or MST2 in the sample biopsy, compared with the control biopsy, is indicative of cancer in the subject. In some embodiments, the cancer is prostate cancer. In some embodiments, the cancer is hormone-refractory metastatic cancer. In some embodiments, the subject is a human.

In certain embodiments, the invention teaches a method of prognosing cancer in a subject including: obtaining a sample biopsy from a subject; determining the expression level of MST1 and/or MST2 in the sample biopsy; and comparing the expression level of MST1 and/or MST2 with the expression level of MST1 and/or MST2 from a control biopsy, wherein a lower level of expression of MST1 and/or MST2 in the sample biopsy, compared to the control biopsy, results in a poor prognosis of cancer in the subject. In some embodiments, the cancer is prostate cancer. In some embodiments, the cancer is hormone-refractory metastatic cancer. In some embodiments, the subject is human.

In some embodiments, the invention teaches a method of suppressing cancer growth in a subject by directly and/or indirectly increasing the expression and/or presence of MST1 and/or MST2 in the subject. In certain embodiments, the cancer is prostate cancer. In certain embodiment, the cancer is hormone-refractory metastatic cancer. In some embodiments, the expression of MST1 and/or MST2 is increased in the tumor cells of the subject. In some embodiments, the subject is a human. In certain embodiments, the invention further includes administering a treatment selected from the group consisting of: brachytherapy, chemotherapy, cryosurgery, hormone therapy, radiation therapy, prostatectomy, and combinations thereof. In certain embodiments, the hormone therapy includes treatment selected from the group consisting of: suppressing a hormone, blocking a hormone and eliminating a hormone. In certain embodiments, the hormone is an androgen. In certain embodiments, the hormone is testosterone. In certain embodiments, the chemotherapy includes administering a chemotherapeutic agent that targets AR and/or PI3K/AKT-mTOR.

In certain embodiments, the invention teaches a method of inhibiting cancer in a subject, by directly and/or indirectly increasing the expression and/or presence of MST1 and/or MST2 in the subject. In certain embodiments, the cancer is prostate cancer.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, MST1 forms a protein complex with AR and antagonizes AR activity. A) Co-immunoprecipitation (co-IP) of ectopically expressed human MST1 and endogenous AR in LNCaP (upper panel) or exogenously expressed human AR in HEK 293 (lower panel) cells. Co-IP and western blots (WB) were performed with corresponding antibodies. B) The AR-responsive PSA promoter reporter (p61-Luc) activity in LNCaP under condition where MST1 was knocked down (upper panel) or enforced (lower panel). An unpaired t-test was conducted to analyze for differences between treatments. *P≦0.02. C) The AR-responsive GRE4-Luc in LNCaP (upper panel) and p61-Luc activity in COS-7 cells (lower panel). Serum starved cells were treated with (+) or without 1 nM R1881, androgen analog, *P≦0.02. All assays were performed at 36 hr. Relative luciferase units as luciferase (Luc) activity after normalization with total protein. Data are the representative of multiple experiments.

FIG. 2 demonstrates, in accordance with an embodiment of the invention, caspase cleavage-deficient MST1 is a potent AR inhibitor. A) AR binding domains on MST1. AR with vector (V), MST1-wt (wild type) or MST1 domains (MST1-N or MST1-C) was transiently co-expressed in COS-7 cells. Co-IP and WB were performed with antibodies to corresponding proteins. B) PSA promoter reporter activity in LNCaP cells with MST1-wt or MST1 truncation mutant in “A”, *P≦0.01; **P≦0.08. C) Western blots of MST1, AR or PSA protein in LNCaP cells transfected with Mock, MST1-wt, MST1-D326N (D/N), MST1-D349E (D/E), or MST1-D326N/D349E (DD/NE), followed by R1881 (+) or vehicle (−) treatment in serum starved conditions. D) PSA promoter reporter activity with caspase-resistant MST1 constructs under the same conditions as described in “C”, *P≦0.01. Luciferase activity was determined at 36 hr. Data are the representative of multiple experiments.

FIG. 3 demonstrates, in accordance with an embodiment of the invention, purified recombinant MST1 binds and phosphorylates GST-AR-D/HR fragment at Ser-650. A-B) GST-pull down in “A” and in vitro kinase assay with bacterially expressed GSTAR-D/HR and recombinant, preactivated MST1 protein kinase in “B, Left panel.” Right panel: WB probed with antibody to MST1 or to phospho-S650 AR in “B”. AR-D/HR: AR-DNA Binding Domain/Hinge Region. C) pGRE4-Luc promoter luciferase activity in COS-7 cells. Cells were co-transfected with pGRE4-Luc and corresponding constructs and treated with R1881 (+) or vehicle (−) in serum starved conditions, *, ** P≦0.01. Luciferase reporter assay was performed at 36 hr following androgen treatment. D) Immunofluorescence staining of AR protein (FITC, cytoplasm with EtOH vehicle and nuclear with R1881) with anti-AR antibody and DAPI (nucleus) in COS-7 cells transfected to express transient AR and stimulated with R1881 (+) or vehicle (−) for 6 hr in serum-starved conditions. The size bar represents 10 microns. Data are representative of multiple experiments.

FIG. 4 demonstrates, in accordance with an embodiment of the invention, the kinase or apoptotic function of MST1 is not involved in AR inhibition. A) Apoptosis in LNCaP or in COS-7 cells transfected with MST1-wt or kinase deficient-MST1 mutant construct (MST1-K59R or MST1-T183A). Cell apoptosis was determined using Cell Death ELISA. B) PSA promoter reporter activity in LNCaP cells with kinase intact or kinase-deficient MST1 as in “A”, followed by incubation with (+) or without (−) R1881. C) MST1 and AR protein complex in COS-7 cells expressing AR along with vector, MST-wt, or kinase-deficient MST1 mutant. Co-IP and western blots were performed in total cell lysates with antibodies to corresponding proteins. D) MST2-regulated PSA promoter reporter activity in LNCaP cells, *P≦0.01. p61-Luc (+) was cotransfected with MST2-wt or with kinase-deficient MST2 mutant (MST2-K56R), *P≦0.01. All assays were conducted at 36 hr. HC: IgG-heavy chain. Data are representative of multiple experiments.

FIG. 5 demonstrates, in accordance with an embodiment of the invention, MST1 antagonizes AKT-mediated AR activation and localizes to AR chromatin complexes. A) Graph: PSA promoter reporter activity modulated with MST1 and AKT signaling, *, **P≦0.01. LNCaP cells were transiently co-transfected with p61-luc promoter reporter and AKT1 or MST1 alone or together with AKT1 and MST1, followed by androgen or vehicle treatment in serum-starved conditions. Blots: A tri-partite protein complex between AR, MST1 and AKT1 in COS-7 cells expressing transient MST1 along with AR or AR and AKT1. All assays were conducted at 36 hr. HC: IgG-heavy chain. B) Co-IP and WB analysis of protein complexes between endogenous AR and MST1 in LNCaP cells grown in basal condition. C-D) ChIP assay in lysates of LNCaP cells. Protein-DNA complexes were precipitated with IgG (negative control), Pol II (positive control), AR, or MST1 antibody in total cell lysates. DNA interaction was assessed by semi-quantitative PCR using primers surrounding AREIII within the androgen-responsive element enhancer core (AREc) and surrounding AREI and TATA region of the PSA promoter. Diagram showing AREc and proximal PSA promoter region where multiple AREs and TATA box, AREI, and AREII reside, respectively. Primer pair against PSA gene was used in input control. Data are representative of multiple experiments.

FIG. 6 demonstrates, in accordance with an embodiment of the invention, enforced MST1 expression suppresses prostate tumor cell growth in monolayer culture and colony formation on soft agar. A) Cell proliferation with BrdU incorporation assay, *P≦0.2; **P≦0.0009. B) Stable MST1 expression by western. LNCaP/HA-MST1 and C4-2/HA-MST1 cells treated with increasing doses (0, 0.5, 1, 2, and 4 μg/ml) of Dox (Doxycycline), respectively, in “A” and “B”. Western blots probed with anti-HA antibody or anti-beta actin as a loading control. C) Colony formation assay on soft agar. Equal numbers of C4-2/HA-MST1 cells were seeded on soft agar and grown for 14 days in the presence (0.5 μg/ml) and absence (−) of Dox in regular growth conditions with the media was replaced every 3 days; *P≦0.004. The graph represents the quantification of colonies and the micrograph represents colonies formed on soft agar. D) The growth of C4-2/HA-MST1 cells treated with LY294002 (20 μM), a potent PI3K inhibitor or vehicle (DMSO) in the presence (+) and absence (−) of Dox, *P≦0.08; **P≦0.007. All cell proliferation was determined at 24 hr. Data are representative of multiple experiments.

FIG. 7 demonstrates, in accordance with an embodiment of the invention, enforced MST1 expression suppresses prostate cancer xenografts. A) Left panel: WB analysis of ectopically expressed HA-MST1 in cytoplasmic and nuclear cell fractions obtained from C4-2/HA-MST1 cells treated with Dox (−) or Dox (+). HA-MST1 was probed with anti-HA antibody. Lamin A/C was used as a nuclear extraction control. Right panel: PSA promoter reporter (+) activity in C4-2/HA-MST1 cell treated with (+) or without (−) Dox and vehicle (EtOH) or androgen (R188) at 24 hr following treatment, *P≦0.01. B) ChIP assay in lysates from C4-2/HA-MST1 cells, respectively. Protein-DNA complexes were precipitated with HA-tag antibody. DNA interaction was assessed by semi-quantitative PCR using primers surrounding AREIII within the androgen-responsive element enhancer core (AREc) region of the PSA promoter. Primer pair against PSA gene was used in input control. C) Left panel: Immunohistochemical staining of HA-MST1 with anti-HA antibody in histological sections from tissue xenografts of C4-2/Vector or C4-2/HA-MST1 tumor. The size bar in “C, left panel” represents 100 microns. Right panel: The quantification of subcutaneous tumor growth in mice. The micrograph is representing the tumor mass. 10 mice per group were used. 8 out of 10 mice for C4-2/Vector or 2 out of 10 mice for C4-2/HA-MST1 were developed tumors 12 weeks after subcutaneous inoculation of the cell. Data are representative of multiple experiments. D) Diagram showing the points at which MST1 negatively regulates AR-mediated gene expression.

FIG. 8 demonstrates, in accordance with an embodiment of the invention, MST1 co-precipitated with AR protein complex from PC3-hisAR cells. A) Western blot (WB) analysis of AR in total lysates (lane 1) and purified AR in Ni-NTA complexes (lane 2). B) WB of endogenous MST1 (bottom panel) in the AR protein (upper panel) complexes obtained by Ni-NTA column. Ni-NTA column purification was conducted at room temperature. WB was performed with antibodies to corresponding proteins.

FIG. 9 demonstrates, in accordance with an embodiment of the invention, the full-length MST1 binds and inhibits AR activity. A) PSA promoter reporter activity in LNCaP cells transfected with p61-Luc and vector or MST1, followed incubation with increasing doses (0, 10, or 100 μM) of caspase-3/7 inhibitor in the presence of R1881 (+) or vehicle (−) in serum-starved conditions. Luciferase activity was determined at 36 hr; *P≦0.009. B) Co-IP of AR and MST1-wt or caspase-resistant MST1 (DD/NE mutant) in HEK 293 cells. C) Endogenous phospho- or total-JNK1 or exogenous MST1 protein levels in LNCaP cells. Cells were transfected with mock, MST1-wt, and a single or double caspase-resistant MST1 mutant construct, followed by minus (−) or plus (+) phorbol ester (TPA) treatment in serum starved conditions. The blots with antibodies to corresponding proteins were performed. Data are representative of multiple experiments.

FIG. 10 demonstrates, in accordance with an embodiment of the invention, phospho-S650 has no effect on MST1-mediated AR inhibition or the interaction between the two proteins. A) GST-pulldown experiment with purified, recombinant GST-AR-D/HR fragment and increasing doses of purified, recombinant MST1 protein kinase. B) In vitro kinase assay with increasing volumes of GST-AR-D/HR beads and recombinant, pre-activated MST1protein kinase. 32P-γ-ATP and autoradiography were performed to visualize the phospho-AR signal. C) PSA promoter reporter activity in COS-7 cells transiently co-transfected with p61-Luc, vector, AR-wt or phosphorylation-inactivating mutant and vector or MSTwt, followed by R1881 (+) or vehicle (−) treatment in serum starved conditions, *P≦0.01. D) Co-IP/WB analysis of AR and MST1 in total lysates of COS-7 cells. MST1-wt was coexpressed with Mock (Vec), AR-wt, AR-S650A, or AR-S650D mutant. Experiments in “C” or “D” were performed at 36h following transfection. IP and western blots were performed with corresponding antibodies.

FIG. 11 demonstrates, in accordance with an embodiment of the invention, AR-dependent PSA promoter reporter activity in the presence and absence of MST1 expression. A) PSA promoter reporter activity in COS-7 cells transiently co-transfected with p61-Luc, vector, AR-wt or phosphorylation-inactivating AR mutant, followed by R1881 (+) or vehicle (−) treatment in serum starved conditions. B) PSA promoter reporter activity in C4-2 cells transiently co-transfected with p61-Luc, vector, MST1-wt and MST1-K59R expression construct. Cells were treated with R1881 (+) or vehicle (−) in serum starved conditions. *P≦0.01 and **P≦0.23 are relative to the wild type. Luciferase reporter assays were performed at 36h following transfection.

FIG. 12 demonstrates, in accordance with an embodiment of the invention, the growth suppression in LNCaP cells is a result of MST1 expression. A) BrdU incorporation assay in LNCaP/Vector (Vec) cells treated with Dox (4 μg/ml) or without Dox (−) for 24 hr. B) The growth of LNCaP/HA-MST1 in (right panel) cells treated with LY294002 (20 μM), a potent PI3K inhibitor or vehicle (DMSO) vehicle in the presence (+) and absence (−) of Dox; *P≦0.001. C) Fluorescence images of HA-MST1 expression probed with anti-HA as a primary and Cy3-labeled (1:2000 dilutions) as a secondary antibody.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed, J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein:

The abbreviation “AR” means androgen receptor.

The abbreviation “ARE” means androgen-responsive element enhancer.

The abbreviation “ChIP” means chromatin-immunoprecipitation.

The abbreviation “Co-IP” means co-immunoprecipitation.

The abbreviation “Dox” means doxycycline.

The abbreviation “GST” means glutathione s-transferase.

The abbreviation “IHC” means immunohistochemistry.

The abbreviation “aJNK1” means c-Jun N-Terminal Protein Kinase 1.

The abbreviation “IPTG” means isopropyl β-D-1-thiogalactopyranosid.

The abbreviation “MAP” means mitogen-activated protein kinase.

The abbreviation “mTOR” means mammalian target of rapamycin.

The abbreviation “PCa” means prostate cancer.

The abbreviation “PI3K” phosphatidylinositol 3-kinase.

The abbreviation “WB” western blot.

The MST1 serine-threonine kinase, a component of the RASSF1-LATS tumor suppressor network, is involved in cell proliferation and apoptosis and has been implicated in cancer. However, the physiologic role of MST1 in prostate cancer is not well understood. The inventors investigated the possibility of a biochemical and functional link between androgen receptor (AR) and MST1 signaling. The inventors showed that MST1 forms a complex with, and antagonizes, AR transcriptional activity as demonstrated by coimmunoprecipitation (co-IP), promoter reporter analysis and molecular genetic methods. In vitro kinase and site-specific mutagenesis approaches indicate that MST1 is a potent AR kinase; however, surprisingly, the kinase activity of MST1 and its pro-apoptotic functions were shown not to be involved in inhibition of AR. MST1 was also found in AR-chromatin complexes, and enforced expression of MST1 reduced the binding of AR to a well-characterized, androgen-responsive region within the prostate specific antigen (PSA) promoter. MST1 suppressed prostate cancer cell growth in vitro and tumor growth in mice. While not wishing to be bound by any one particular theory, because MST1 is also involved in regulating the AKT1 pathway, this kinase may be an important new link between androgenic and growth factor signaling and a novel therapeutic target in prostate cancer.

Furthermore, the inventors provide evidence that enforced MST1 expression sensitized androgen-independent C4-2 cells to PI3K inhibition. While not wishing to be bound by any one particular theory, these findings strongly suggest that loss of MST1 signaling may promote hyperactivation of AR and may be associated with the emergence of the castration-resistant phenotype.

In certain embodiments, the invention teaches a method of suppressing cancer growth in a subject by directly and/or indirectly increasing the expression and/or presence of MST1 and/or MST2 in the subject. In certain embodiments, a compound is administered that increases MST1 and/or MST2 expression, directly or indirectly. One of skill in the art would readily appreciate there are many ways to increase the expression of MST1 and/or MST2 in a subject. In certain embodiments, expression is increased by administering a vector designed to increase the expression of MST1 and/or MST2 in a subject. In certain embodiments, MST1 and/or MST2 are administered directly to the subject as therapeutic molecules by engineering them into a delivery system and/or linking them to carriers in a tissue specific fashion. In some embodiments, the cancer is prostate cancer. In certain embodiments, the cancer is hormone-refractory metastatic cancer. In certain embodiments, the expression of MST1 and/or MST2 is increased in the tumor cells of the subject. In some embodiments, the subject is an animal. In certain embodiments, the subject is a mammal. In some embodiments, the subject is a human. In some embodiments, the invention further includes administering a treatment selected from the group including: brachytherapy, chemotherapy, cryosurgery, hormone therapy, radiation therapy, prostatectomy, and combinations thereof. In certain embodiments, the hormone therapy includes treatment selected from the group consisting of: suppressing a hormone, blocking a hormone, and eliminating a hormone. In certain embodiments, the hormone is an androgen. In certain embodiments, the hormone is testosterone. In some embodiments, the chemotherapy comprises administering a chemotherapeutic agent that targets AR and/or PI3K/AKT-mTOR.

In various embodiments, the invention teaches a method of inhibiting cancer in a subject, by directly and/or indirectly increasing the expression and/or presence of MST1 and/or MST2 in the subject. One of skill in the art would readily appreciate there are many ways to increase the expression of MST1 and/or MST2 in a subject. In certain embodiments, expression is increased by administering a vector designed to increase the expression of MST1 and/or MST2 in a subject. In certain embodiments, MST1 and/or MST2 are administered directly to the subject as therapeutic molecules. In certain embodiments, the expression and/or presence of MST1 and/or MST2 is increased in tumor cells of the subject. In certain embodiments, the cancer is prostate cancer. In some embodiments, the cancer is hormone-refractory metastatic cancer.

In certain embodiments, the invention teaches a method of detecting cancer in a subject, including: obtaining a sample biopsy from a subject; determining the expression level of MST1 and/or MST2 in the sample biopsy; and comparing the expression level of MST1 and/or MST2 with the expression level of MST1 and/or MST2 from a control biopsy; wherein a lower level of expression of MST1 and/or MST2 in the sample biopsy, compared with the control biopsy, is indicative of cancer in the subject. In certain embodiments, the cancer is prostate cancer. In certain embodiments, the cancer has progressed to the castration-resistant metastatic state. In some embodiments, the subject is an animal. In certain embodiments, the subject is a mammal. In some embodiments, the subject is a human.

In certain embodiments, the invention teaches a method of prognosing cancer in a subject, including: obtaining a sample biopsy from a subject; determining the expression level(s) of MST1 and/or MST2 in the sample biopsy; and comparing the expression level(s) of MST1 and/or MST2 with the expression level(s) of MST1 and/or MST2 from a control biopsy, wherein a lower level of expression of MST1 and/or MST2 in the sample biopsy, compared to the control biopsy, results in a poor prognosis of cancer in the subject. In certain embodiments, the cancer is prostate cancer. In certain embodiments, the cancer is hormone-refractory metastatic cancer.

In certain embodiments, the invention teaches a method of screening for compounds that regulate levels of MST1 and/or MST2, directly or indirectly. In certain embodiments, the levels of MST1 and/or MST2 are determined before and/or after administering a compound to a subject. In certain embodiments, in vitro experiments are performed to determine the effect of a compound on MST1 and or MST2 expression. In certain embodiments, MST1 levels are determined using the antibodies of the present invention, according to one or more of the inventive methods for visualizing detection described herein.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive. One skilled in the art will recognize many methods and materials similar or equivalent to those described herein, which could be used in the practice of the present invention. Indeed, the present invention is in no way limited to the methods and materials described. Various embodiments of the invention are described above in the Description of the Invention. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

### EXAMPLES

### Example 1

### Plasmid Constructions, Antibodies and Reagents

The construction of HA- or Myc-tagged MST1-wt and Myc-MST1-N and Myc-MST1-C forms was described previously (15). For the construction of Doxyclineinducible HA-MST1 plasmid, PCR-amplified HA-tagged MST1-wt cDNA was inserted into BamH1 and M/u/enzyme sites in the pRetro-X-Pur vector (Clontech Laboratories, Inc., Mountain View, Calif.), designated as pRXTP-HA-MST1. The construction of GST-AR DBD/HR (AR DNA binding domain and hinge region) was described previously (17). MST1 and AR point mutations were generated using the QuickChange site-directed mutagenesis kit (Stratagene, La Jolla, Calif.). The orientation and fidelity of all constructs were confirmed by DNA sequencing.

### Example 2

### Cell Transfections, Reporter Assays, and Immunocytochemistry

LNCaP and C4-2 were cultured in RPMI 1640 medium and HEK 293T and COS-7 cells were cultured in DMEM at 37° C. in 5% CO2 incubator. Media were supplemented with 10% FBS and 1% penicillin/streptomycin. RNAi (siRNA) transfections with DharmaFECT 2 and plasmids transfections with Lipofectamine 2000 were performed according to the manufacturer's instructions (Invitrogen). Luciferase reporter gene activities were measured using the Luciferase Assay System from Promega (Madison, Wis.) and a BMG Labtech microplate reader (Cary, N.C.). Relative luciferase units were normalized to total protein and the result presented as luciferase (Luc) activity. Immunocytochemistry was performed as described previously (15). Cells were imaged at 63× with a Plan-Apochromat oil immersion lens on an Axioplan 2 Apotome epifluorescence microscope (Zeiss, Germany). Immunohistochemistry (IHC) was performed using reagents from DAKO (Carpinteria, CA) and images were acquired at 20× with Nikon Imaging System (Japan).

### Example 3

### Establishment of TetON-Inducible Cells

Retroviruses carrying Tet-repressor or HA-MST1 expression constructs were produced in HEK 293T cells expressing viral packaging proteins and then viral particles were concentrated using PEG-it solution. LNCaP parental cells or its castration-resistant subline, C4-2, were first infected with retrovirus encoding pRetroX-TetON advanced plasmid, followed by selection with Geneticin (G418, 500 μg/ml) to generate the TetON cells. The LNCaP/ or C4-2/TetON cells were then infected with retrovirus encoding pRXTP-HA-MST1 vector, followed by Puromycin selection (3 μg/ml) to generate TetON inducible MST1 expressing cells. The inducible system allows fine control of MST1 expression. All protocols and procedures were performed according to the manufacturer's instructions (Clontech Laboratories, Inc., Mountain View, Calif.).

### Example 4

### Protein Analyses

Cell lysis was performed in buffer consisting of 20 mM HEPES, pH 7.4, 150 mM NaCl, 0.5% NP-40, 1 mM EDTA, protease inhibitors and phosphatase inhibitor. For immunoprecipitation, cleared lysates were incubated with antibody overnight at 4° C. Antibody-antigen complexes were collected on Protein A- or G-sepharose and washed three times with cell lysis buffer. Immunoprecipitates were resolved by SDS-PAGE. PBST (0.1% Tween-20) containing 5% (w/v) skim milk powder or PBST containing 5% IgG free BSA (Sigma) was used in membrane blocking and antibody dilutions. Signals were visualized by chemiluminescence. GST-AR DBD/HR and its mutant forms were expressed in bacteria with IPTG induction and protein purification was performed with GST-sepharose using a standard protocol. Cytoplasmic and nuclear fractions were prepared as described (18).

### Example 5

### ChIP Assays

Chromatin Immunoprecipitation (ChIP) was performed as described previously (19). Briefly, LNCaP or C4-2/HA-MST1 cells grown in serum-starved conditions were treated with R1881 (1 nM) or EtOH (vehicle) overnight. Dox −/+ used to induce MST1 expression in C4-2/HA-MST1 cells. DNA enriched with anti-MST1, -AR, or -Pol II antibody were quantified by semi-quantitative PCR using primer sets surrounding the AREIII region within the androgen responsive element enhancer (ARE) core (AREc) or AREI of the PSA promoter (18).

### Example 6

### Kinase, Cell Death, and Cell Proliferation Assays

For in vitro kinase assay, the recombinant, pre-activated MST1 protein kinase was incubated with purified GST-AR DBD/HR fusion protein and 10 μCi ³²P-γ-ATP or 100 μM unlabeled-ATP. The reaction mixture was resolved on SDS-PAGE and autoradiographed. Cell-Death ELISA and BrdU incorporation assays were performed to assess cell death and cell proliferation, respectively, according to the manufacturer's instructions (Roche Molecular Diagnostics).

### Example 7

### Animal Studies

C4-2/Vector or C4-2/HA-MST1 cells mixed with Matrigel (1-to-1 ratio) were inoculated subcutaneously in athymic nude mice (CD-1 nu/nu; Charles River Laboratories, Wilmington, Mass.). 1×10⁶ cells/100 μl were used per injection per site (right and left flanks). Mice were treated with Dox (0.5 mg/ml) in drinking water to induce MST1 expression for 12 weeks. Institutional Animal Care and Use Committee (IACUC) policies and guidelines were strictly applied. Tumor volumes were assessed by caliper according to procedures described previously (20). Mice were sacrificed and evaluated for tumor growth anatomically and tumor tissues extracted from mice were fixed in 10% formaldehyde for the construction of histological sections or “snap” frozen at −80° C. The expression of MST1 was verified in histological sections by immunohistochemistry using anti-HA antibody.

### Example 8

### Statistics

Data are represented as mean +/− SEM. Student t-test (2-tailed) was used between the data pairs where it is appropriate. A p-value less than or equal to 0.05 was considered significant.

### Example 9

### MST1 Binds and Attenuates AR Activity

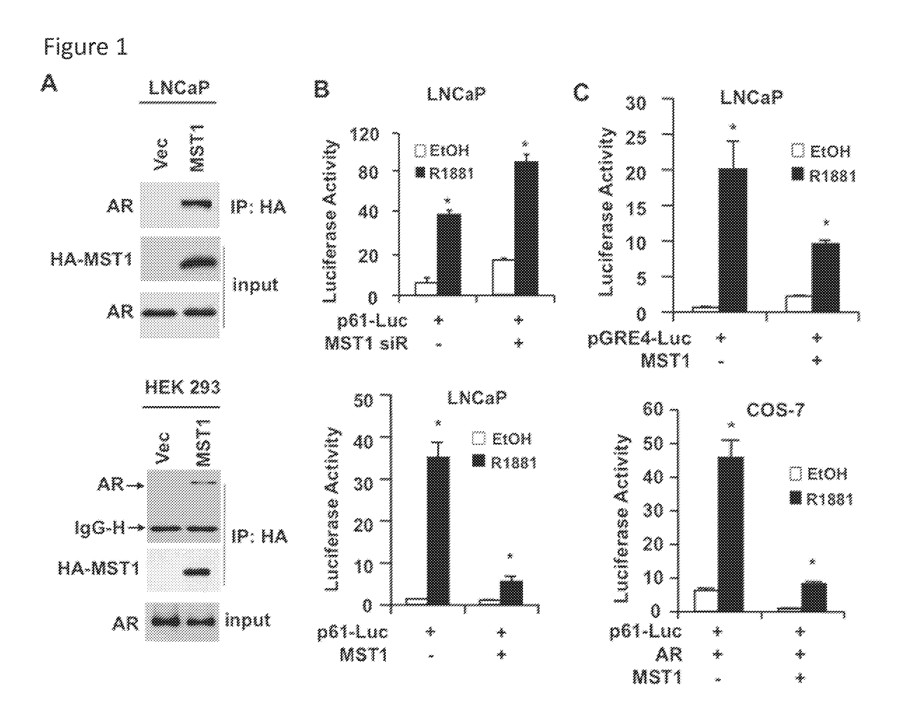

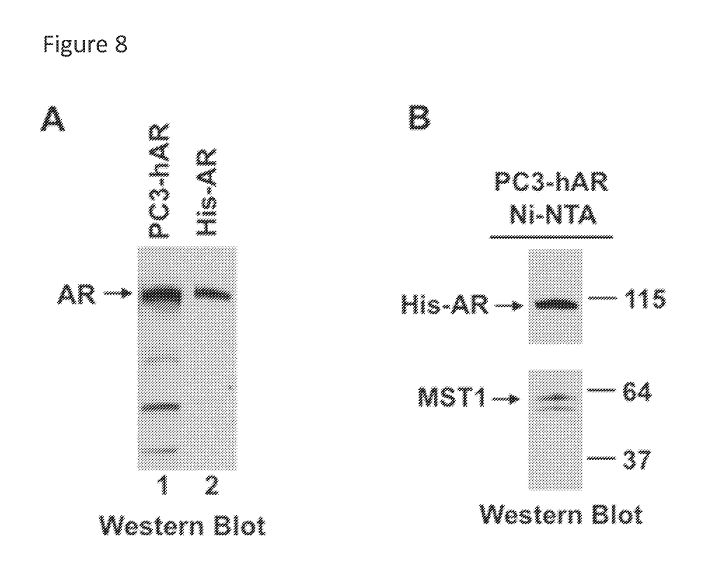

To determine whether the MST1 kinase biochemically and functionally intersects with AR signaling, the inventors employed cellular and biochemical approaches using prostate and non-prostate cancer cell lines that express either native, stable or transient AR. Coimmunoprecipitation (Co-IP) and western blot experiments demonstrated that MST1 interacts with endogenous AR in LNCaP cells (FIG. 1A, upper panel) and ectopically expressed human AR in HEK 293 cells (FIG. 1A, lower panel). Similarly, endogenous MST1 could also be found in the AR protein complex from PC3-hAR cells that were engineered to express near-physiological levels of stable his-tagged human AR (18), as shown by Ni-NTA precipitation and western blot experiments (FIG. 8A and FIG. 8B).

To test whether the binding of MST1 has an impact on AR activity, the inventors conducted AR-dependent promoter-reporter assays (18, 21) using knockdown and induction approaches. MST1 knockdown by a gene-specific small interfering RNA (siRNA) (15) resulted in the upregulation of basal and androgen-stimulated AR-responsive PSA promoter reporter activation, by at least 2-fold in comparison to siRNA control (FIG. 1B, upper panel). Enforced MST1 expression attenuated endogenous AR activity in LNCaP cells, as shown by the PSA promoter reporter (p61-Luc) assay (FIG. 1B, lower panel). Enforced MST1 expression also attenuated AR-dependent GRE4-driven simple promoter reporter (pGRE4-TATA-Luc) activation in LNCaP (FIG. 1C, upper panel) as well as p61-Luc promoter reporter activation in COS-7 (FIG. 1C, lower panel) cells, where AR expression was also enforced. Collectively, these observations indicate that MST1 is a binding partner and a physiologic negative regulator of AR signaling.

### Example 10

### The Full-Length MST1 is a Dominant AR Suppressor

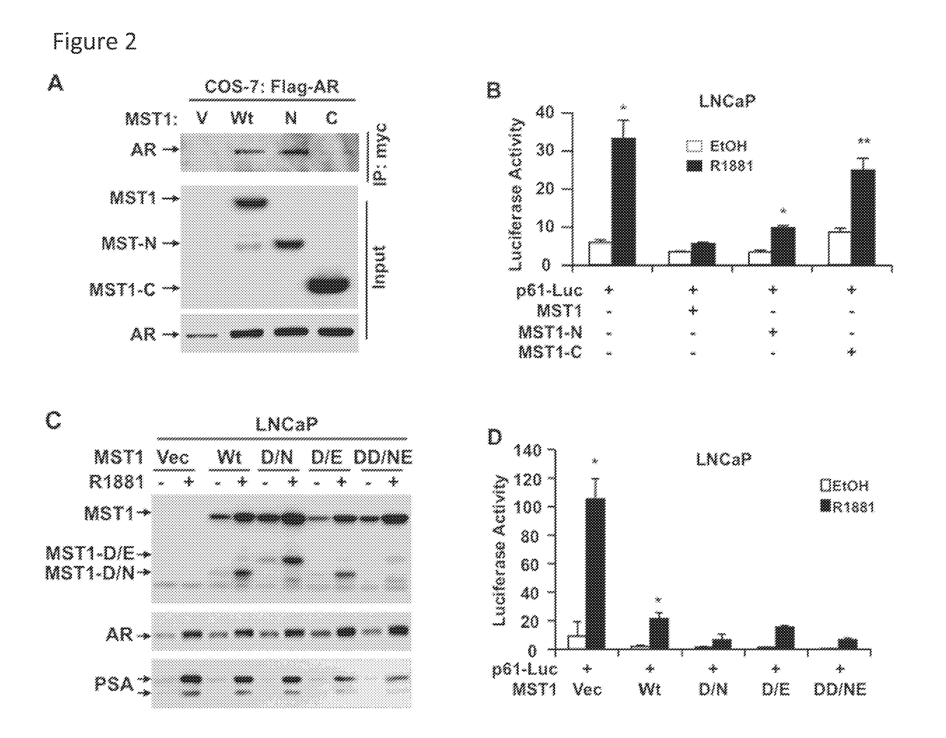

To map the AR binding domain on MST1, the full-length AR was co-expressed with vector, MST1-wt or MST1-N (residues 1-330) or MST1-C (residues 331-487) truncation mutants in COS-7 cells, followed by co-IP and western blot analysis. The results showed that the full-length MST1 (MST1-wt) and MST1-N strongly interacted, whereas MST1-C displayed weak interaction with exogenous AR (FIG. 2A). The inhibition of AR activity by MST1-wt, MST1-N or MST1-C coincided with the binding data, as revealed by the PSA promoter reporter assay (FIG. 2B).

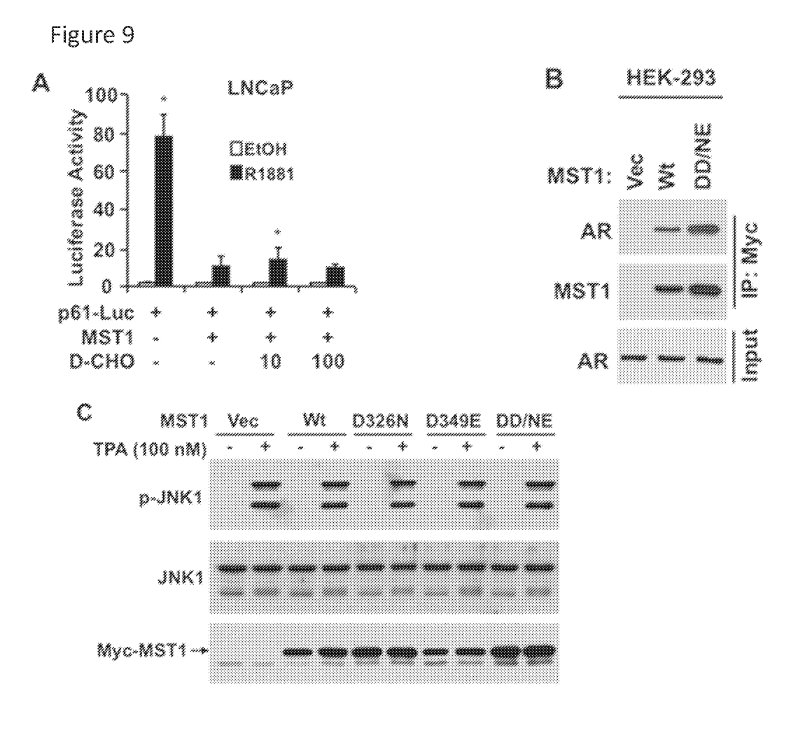

To determine which MST1 form (MST1-wt or the cleaved MST1-N) functions as a dominant AR inhibitor, the inventors generated caspase-resistant single (D326N=D/N, Asp (D)→Asn (N) or D349E=D/E, Asp→Glu (E)) and double (D326N/D349E=DD/NE) MST1 mutants and assessed their effects on PSA protein levels or luciferase reporter activity mediated by AR. The results show (FIGS. 2C and 2D) that the expression of each MST1 mutant is capable of inhibiting endogenous PSA protein levels and PSA promoter reporter activation induced by androgen, similar to the levels seen with MST1-wt. Neither the expression of MST1-wt nor these MST1 mutants affected AR protein levels, unless the cells were stimulated by androgen (FIG. 2C, AR blot). A similar level of AR-dependent PSA promoter reporter inactivation by MST1 was also obtained, even in the presence of increasing doses of a specific caspase inhibitor, Ac-DEVD-CHO (DCHO) (FIG. 9A).

The inventors then examined protein complexes between AR and the caspase-deficient MST1-DD/NE mutant. AR was co-expressed with vector, MST1-wt, or MST1-DD/NE mutant constructs in HEK 293 cells. As shown by co-IP/western blot experiments, the MST1-DD/NE mutant maintained interaction with AR and the levels of interaction between AR and MST1-DD/NE were similar or even greater than that observed with MST1-wt (FIG. 9B). These observations indicate that the caspase deficient MST1 is a dominant AR inhibitor.

### Example 11

### MST1 Attenuates AR Activity in a Ser-650 phosphorylation-Independent Manner

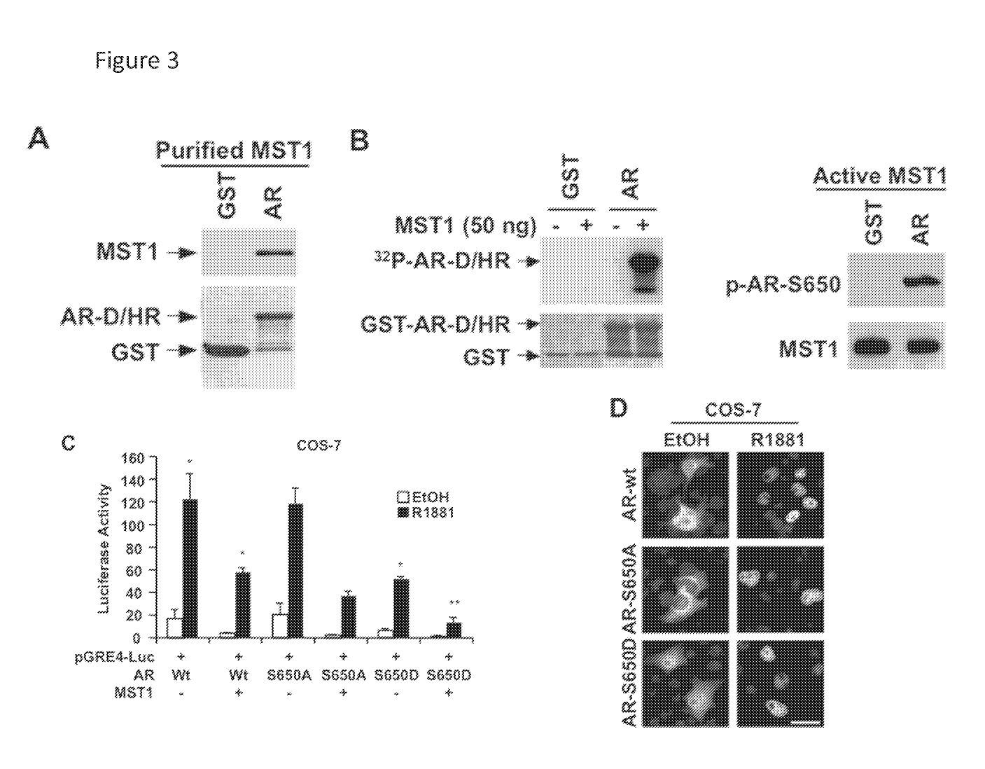

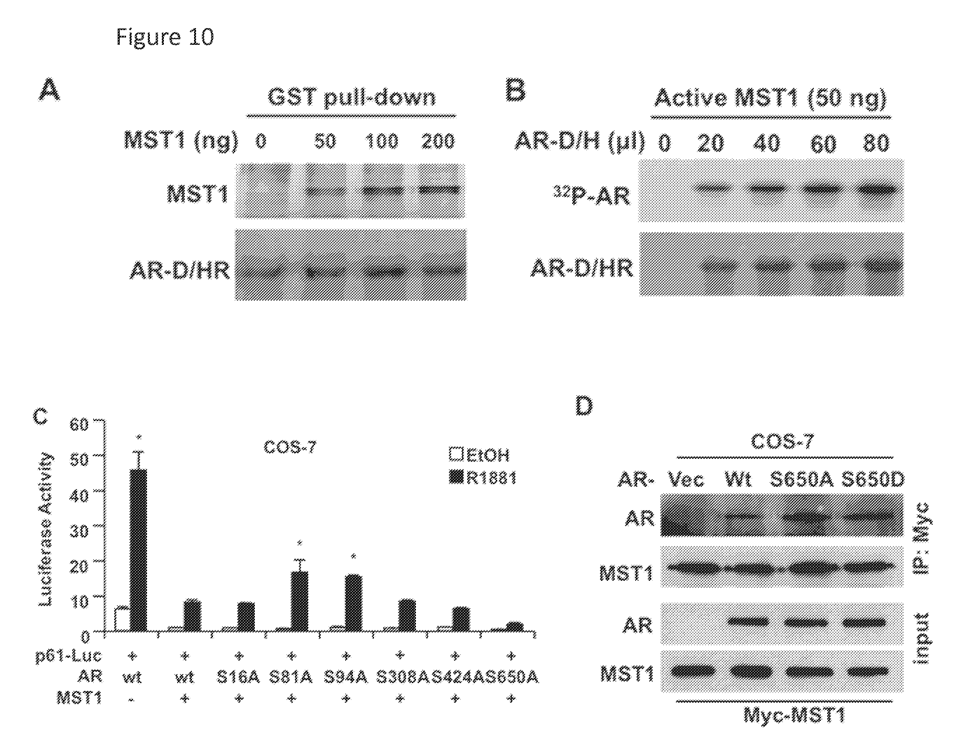

MST1 is a stress-induced kinase (2), and other stress-induced kinases such as JNK1 or p38 MAPK have been proposed to physically interact with and antagonize AR transcriptional activity by phosphorylating AR at Ser-650 (17). To determine whether purified MST1 can also physically interact with and phosphorylate AR at this site, the inventors performed the GST-pull down and an in vitro kinase assay using recombinant, preactivated MST1 and purified GST-AR-DBD/HR as a substrate. The results of these experiments revealed that pre-activated, recombinant MST1 physically interacted with (FIG. 3A) and specifically phosphorylated the purified GST-AR-DBD/HR fragment in vitro (FIG. 3B, left panel). Both binding and phosphorylation events occurred in a dose dependent manner (FIGS. 10A and 10B, respectively). Using a site specific phospho-AR antibody, the inventors were able to identify the Ser-650 residue as a target of the MST1 kinase activity, as revealed by non-radioactive in vitro kinase assay and western blot experiments (FIG. 3B, right panel).

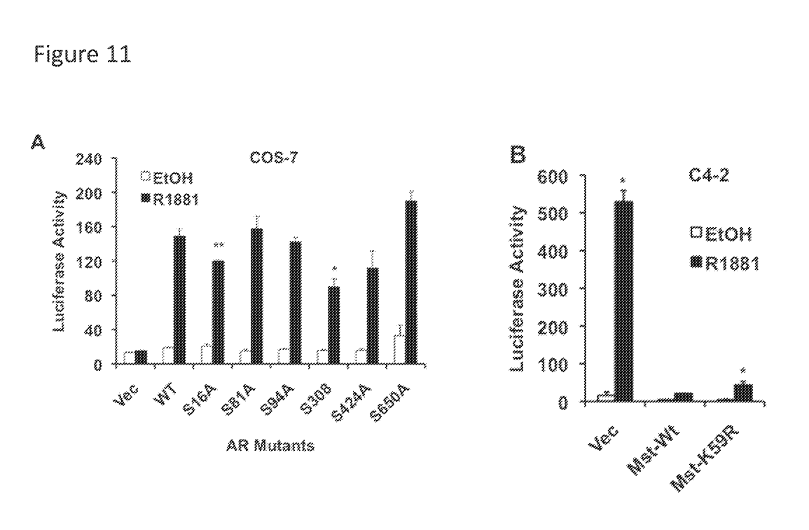

To determine whether Ser-650 phosphorylation has a role in the attenuation of AR activity by MST1, the inventors generated phosphorylation-inactivating (Ser→Ala) or phosphomimetic (Ser→Glu) mutations and assessed their impact on MST1-mediated inhibition of AR activity. The data in FIG. 3C show that enforced-MST1 expression is capable of inhibiting AR transcriptional activity regardless of the presence of phosphorylation-inactivating S650A or phosphomimetic S650D AR mutations. Similarly, other site-specific phosphorylation-inactivating mutations did not significantly affect AR inhibition by MST1 induction (FIG. 10C). In addition, phosphorylation inactivating or phosphomimetic mutations did not alter ligand-dependent nuclear localization of AR in COS-7 cells expressing exogenous AR (FIG. 3D). Furthermore, co-IP/western blot analyses showed that neither type of mutation affected complex formation between the two proteins (FIG. 10D). In addition, with the exception of the S308A mutation, these known phospho-site mutations do not significantly alter androgen-induced AR transcriptional activity in COS-7 cells (FIG. 11A). While not wishing to be bound by any one particular theory, these findings appear to indicate that the phosphorylation of Ser-650 by MST1 is not involved in the mediation of AR inhibition by MST1.

### Example 12

### MST1 Kinase Activity is not Required for the Inhibition of AR Activity

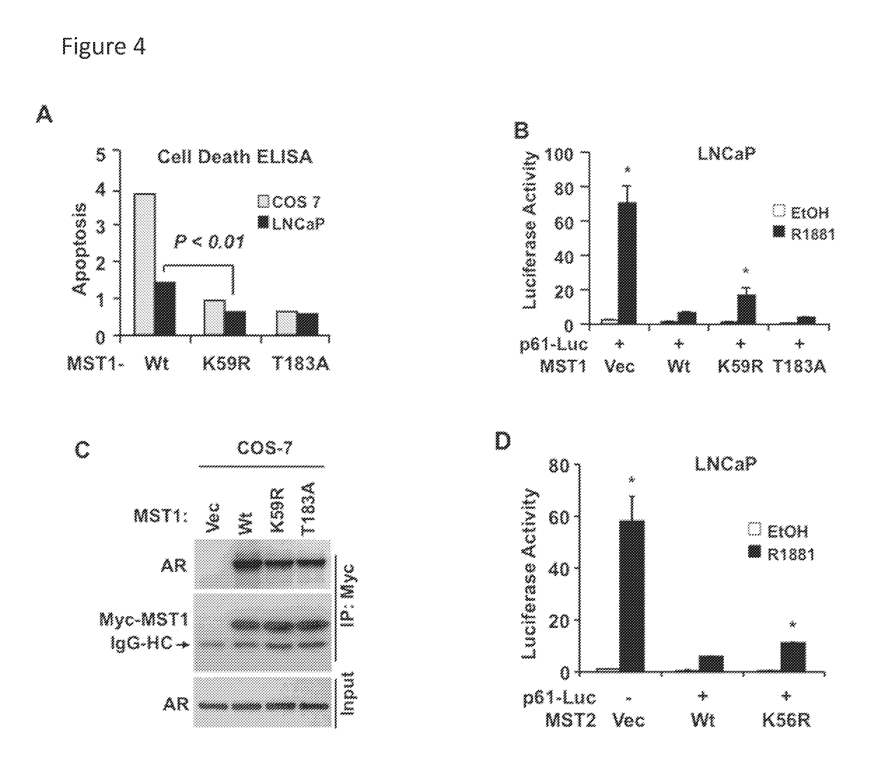

To determine whether MST1 kinase activity has an effect on the inhibition of AR transcriptional activity, the inventors generated kinase-deficient MST1 mutants (MST1-K59R in the ATP binding pocket and MST1-T183A in the activation loop). Consistent with published data (3), neither of these MST1 mutants was able to induce apoptosis in COS-7 or in LNCaP cells, compared to MST1-wt (FIG. 4A). However, both mutants were capable of inhibiting AR-driven PSA promoter activation, similar to the levels observed with MST1-wt in LNCaP (FIG. 4B) and in its castration-resistant C4-2 subline (FIG. 11B). The inventors then examined the protein complexes between AR and these kinase-inactivated MST1 mutants. Mock, MST1-wt, MST1-K59R, or MST1-T183A constructs were co-expressed with AR in COS-7 cells and co-IP experiments were performed using total cell lysates. Western blot analysis with anti-AR antibody revealed that the kinase-inactivating mutation had no affect on the formation of the AR and MST1 complex (FIG. 4C). The inventors also performed the promoter reporter assay with MST2 and demonstrated that transient expression of this close structural relative to MST1 also antagonized the AR transactivation function in LNCaP cells independently of its kinase activity (FIG. 4D). While not wishing to be bound by any one particular theory, these results suggest that both kinases perform a similar inhibitory role with respect to the AR, and that neither the kinase activity nor the proapoptotic function of MST1 or MST2 is involved in the inhibition of AR activity.

### Example 13

### MST1 Suppresses AR Activity by Intersecting with AKT1 Signaling and Antagonizing Formation of AR-Chromatin Complexes

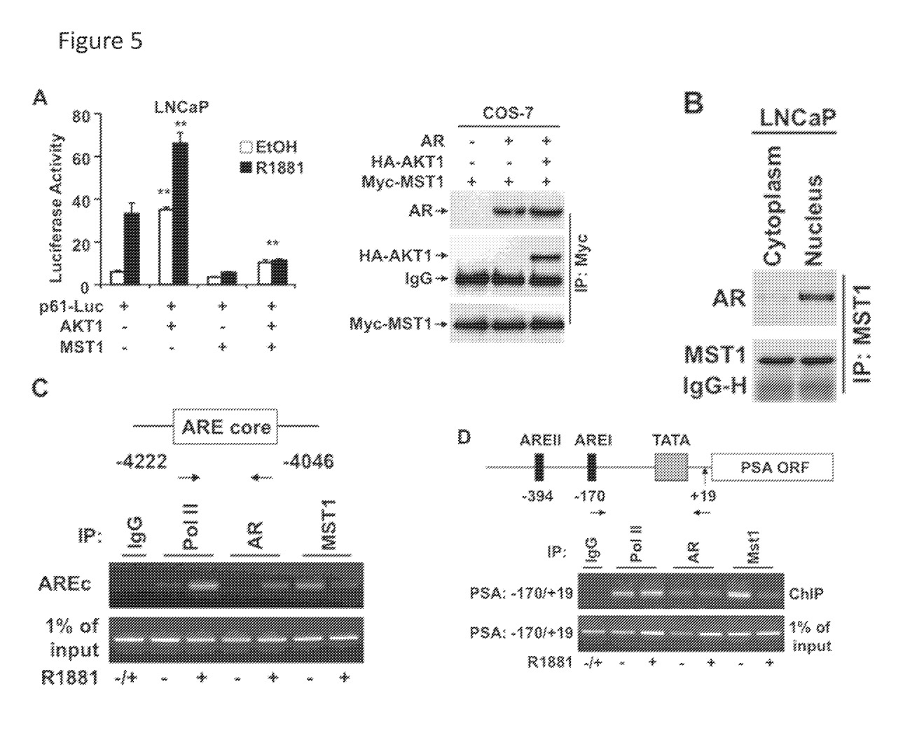

An implication from the above findings is that additional mechanisms may be involved in MST1-mediated AR inhibition. MST1 was reported to inhibit AKT signaling (15), which is known to functionally intersect with (16, 22) and promote AR-driven PSA promoter activation (23). To test whether MST1 induction could suppress AR activation mediated by AKT1 signaling, the inventors performed promoter-reporter assays and showed that enforced MST1 expression antagonized AKT1 mediated androgen-dependent and -independent AR activation (FIG. 5A, left panel). Co-IP experiments further revealed that MST1, AR, and AKT1 form a tri-partite complex in vivo (FIG. 5A, right panel), indicating that MST1 also intersects with AKT signaling to attenuate AR activity.

Given that MST1 forms protein complexes with AR, we showed using co-IP experiments that endogenous AR and MST1 form a complex preferentially in cell nuclei (FIG. 5B). These observations led the inventors to investigate whether MST1 localizes within AR transcriptional complexes. Chromatin immunoprecipitation (ChIP) experiments showed that endogenous MST1 interacts with the PSA promoter region, which also binds AR in both serum- and androgen free conditions and can be regulated by androgen in LNCaP cells (FIG. 5C). This segment of the PSA promoter, referred to as the androgen-responsive element enhancer (ARE) core, consists of multiple AREs and plays a prominent role in AR-driven PSA promoter regulation (18, 21). Additional data suggest that MST1 also binds ARE-I and the TATA region in the PSA promoter, which can also be regulated by androgen (FIG. 5D). While not wishing to be bound by any one particular theory, taken together, these findings suggest that MST1 reduces AR chromatin complex formation to attenuate androgenic signals.

### Example 14

### MST1 Suppresses PCa Cell Growth In Vitro and Tumor Growth In Vivo

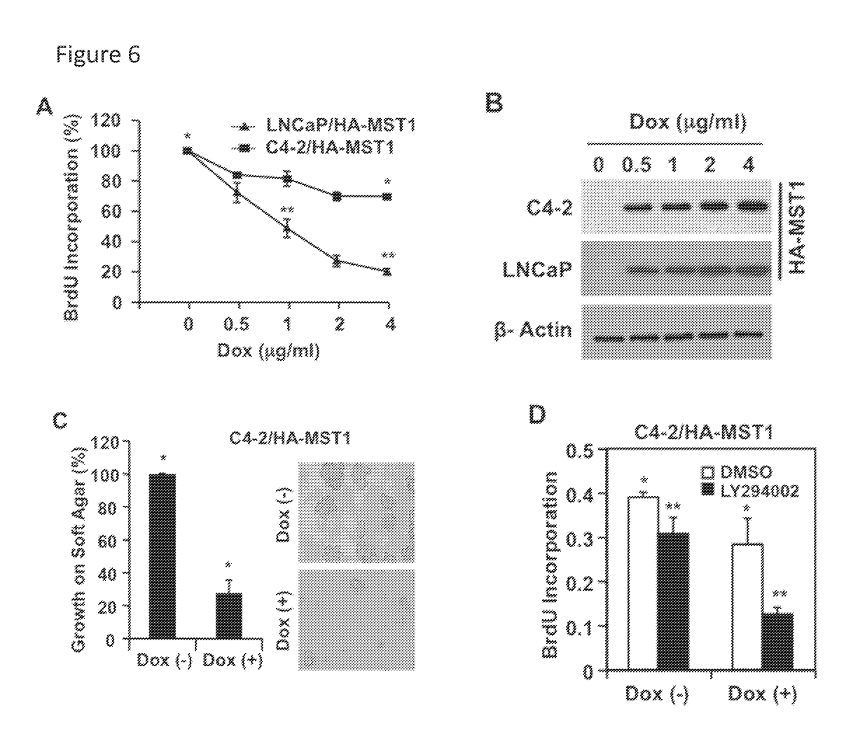

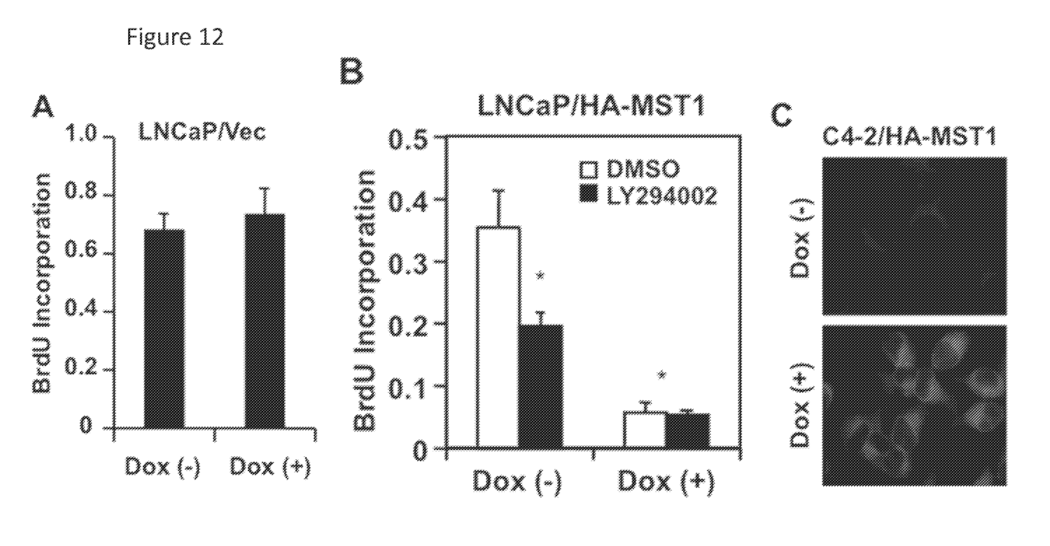

To address the impact of MST1 on cell growth, the inventors established stable MST1 expressing LNCaP/HA-MST1 or C4-2/HA-MST1 cells using a retroviral inducible system. LNCaP/HA-MST1 or C4-2/HA-MST1 cells were exposed to increasing doses of doxycycline (Dox). Dose-dependent induction of MST1 expression reduced the growth of LNCaP; however, castration-resistant LNCaP subline, C4-2, displayed resistance to the growth suppressive effects of enforced MST1 (FIGS. 6A and B). The observed growth reduction originated from MST1 induction because the administration of the highest dose of Dox (4 μg/ml), which reduced the growth of LNCaP/HA-MST1 the most, had no effect on the growth of LNCaP/Vector cells (FIG. 12A). In addition, enforced MST1 expression in C4-2/HA-MST1 cells dramatically reduced the number and size of C4-2 colonies grown on soft agar (FIG. 6C) and sensitized C4-2 cells to growth suppression induced by PI3K inhibitor LY294002 (FIG. 6D). C4-2 cells are relatively resistant to PI3K inhibitors (24) and less sensitive to the effects of MST1 compared to parental LNCaP cells (FIG. 6A). As expected, induction of MST1 prevented the growth of LNCaP cells regardless of the presence of PI3K inhibitor (FIG. 12B).

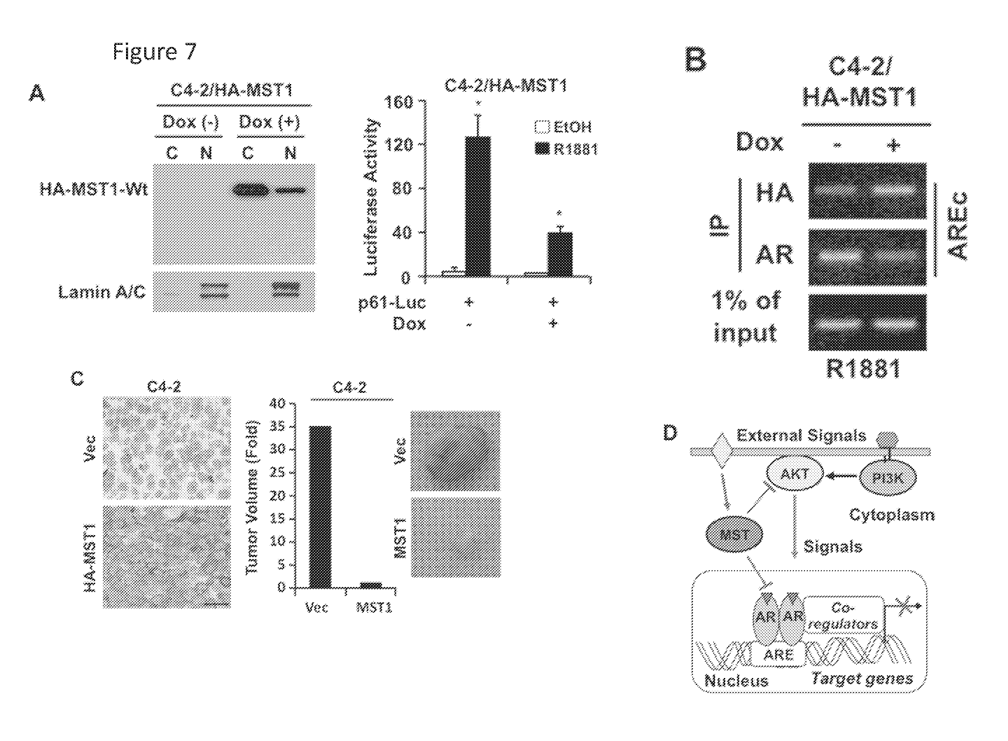

To assess the distribution of MST1, the inventors analyzed levels of exogenous MST1 protein in cytoplasmic and nuclear fractions obtained from C4-2/HA-MST1 cells. The result revealed that although the majority of exogenous HA-MST1-wt localized in the cytoplasm, significant levels of exogenous HA-MST1-wt protein were also found in the nucleus (FIG. 7A, left panel). Immunofluorescence experiments confirmed these results (FIG. 12C). Stable MST1 expression was capable of inhibiting AR driven PSA promoter activation in C4-2/HA-MST1 cells (FIG. 7A, right panel). ChIP experiments using lysates from C4-2/HA-MST1 cells further demonstrated that loading of MST1 onto the ARE core region of the PSA promoter diminished the formation of AR-chromatin complexes (FIG. 7B).

To determine the physiologic relevance of the in vitro findings, the inventors performed xenograft experiments. C4-2/HA-MST1 or C4-2/Vector cells were inoculated subcutaneously into immunodeficient male mice and the animals were then treated with Dox in the drinking water. Immunohistochemical analyses of the resultant tumors verified the expression of HA-MST1 in histological sections from C4-2/HA-MST1 or C4-2/Vector tumor xenografts (FIG. 7C, micrograph). Consistent with observations in vitro, enforced MST1 expression suppressed the growth by 35-fold and the tumorigenic rates by 4-fold of C4-2/HA-MST1 cells compared to C4-2/Vector counterparts (FIG. 7C, graph and tumor mass image).

### Example 15

### Antibodies and Reagents

Antibodies to MST1 from Cell Signaling Technology (Danvers, Mass.), to AR from Millipore (Billerica, Mass.), to HA tag from Covance (Berkeley, Calif.), and to Myc-tag from BD Biosciences (Mountain View, Calif.) were obtained. HRP-conjugated rabbit or mouse secondary antibody was from Pierce (Rockford, Ill.) or from GE Health Care (Piscataway, N.J.) and FITC- or Cy3-labeled secondary antibody was from Jackson ImmunoResearch (West Grove, Pa.). SuperSignal was from Pierce (Rockford, Ill.). DAPI was from Vector laboratories (Burlingame, Calif.). Lipofectamine 2000 was from Invitrogen, Inc (Indianapolis, Ind.). DharmaFECT 2 was from Dharmacon Inc. (Lafayette, Colo.). Caspase-3/7 inhibitor was from EMD (Gibbstown, N.J.). PEG-it virus precipitation solution was from SBI System Biosciences (Mountain View, Calif.). Doxycyline (Dox) was from Sigma (USA). Protein A- or G-sepharose, and GST-sepharose were from GE Health Care (Pasadena, Calif.). Matrigel was from Fisher Scientific or BD Biosciences.

### Example 16

### Discussion

In this study, the inventors demonstrated that the serine-threonine kinase MST1 is a physiologic negative regulator of AR signaling. The inventors provide evidence that MST1 forms protein complexes in vitro and in vivo with AR and antagonizes AR activity in multiple cell backgrounds. Although both the full-length MST1 and the cleaved MST1-N forms bound and inhibited AR activity, caspase-resistant MST1 was the most potent AR inhibitor. The inventors found that the kinase activity of MST1 was not required for the attenuation of AR activity. Similarly, the pro-apoptotic function of MST1 is not involved in AR inhibition because kinase deficient MST1, which failed to induce cell death, was capable of interacting with AR and inhibiting AR transcriptional activity. Furthermore, promoter reporter and ChIP experiments revealed that enforced MST1 antagonized AKT-mediated AR activation and reduced binding of AR to its cognate DNA binding site. While not wishing to be bound by any one particular theory, these observations suggest that MST1 antagonizes AR-dependent gene expression by forming inhibitory protein and/or transcriptional complexes with AR, thereby suppressing prostate tumor growth.

Post-translational modifications such as phosphorylation (17), palmitoylation (25), ubiquitination (26), acetylation (27), or SUMOylation (28) play important roles in the regulation of AR activity. Several phosphorylation sites at serine residues and a tyrosine residue have been identified in AR (29, 30). These modifications negatively or positively regulate AR activity in a context-dependent manner (24), and their functional significance in PCa is beginning to emerge (31). For example, the phosphorylation of AR at Tyr-534 by c-Src was demonstrated to enhance AR activation and AR-dependent gene expression, which was shown be correlated with hormone-refractory PCa (30). On the other hand, phosphorylation of AR at Ser-650 by JNK1 or p38 MAP kinase was demonstrated to inhibit AR activity (17). Here the inventors showed that MST1 is an AR kinase and phosphorylates AR at Ser-650 (FIG. 3C). However, phosphorylation of Ser-650 by MST1 had no effect on the observed inhibition of AR-transcriptional activity. Nevertheless, the role of phospho-Ser650 on mechanisms of AR action deserves further investigation, given that phosphorylation-inactivating S650A or phosphomimetic S650D AR mutations are able to alter the cellular distribution of AR in comparison to that observed with AR-wt under androgen-free conditions (FIG. 3D). The inventors' data do not rule out the possibility that the existence of other potential MST1 phosphorylation sites might play a role in the inhibition of AR activity by MST1. In addition, the induction of MST1 was shown to activate JNK1 (32), which is known to inhibit AR (17). The inventors did not observe JNK1 activation in response to MST1 induction in LNCaP, unless cells were treated with phorbol ester (FIG. 9C). This indicates that MST1 attenuates AR signaling by a distinct mechanism apart from JNK or p38 MAP kinase activation.

In addition to post-translation modifications, transcriptional co-regulators (i.e. co-repressors or co-activators) play an essential role in the modulation of AR activity, and their altered expression has been demonstrated in prostate tumor progression (33). For example, the recruitment of nuclear co-repressor (N-CoR) (34) or silencing mediator for thyroid and retinoid receptors (SMRT) (35) into the AR transcriptional complex was shown to antagonize AR activity by a mechanism involving protein-protein interaction. Similarly, displacement of co-activators such as p300 from the holo-AR transcriptional complex, or recruitment to the complex of histone deacetylase (HDAC), which modifies chromatin structure to a transcriptionally inactive form (36), have also been shown to attenuate AR activity (35). Given that MST1 attenuates AR activity by forming protein complexes, the localization of MST1 into the holo-AR transcriptional complex. However, comprehensive studies are needed to elucidate precisely how MST1 alters AR chromatin complexes.

MST1 and its downstream effectors, such as WW45 or LATS1/2, have been implicated in cancer, including PCa (37). For example, the liver specific knockout of MST1/2 expression in mice has been associated with hepatocellular carcinoma, which has been linked to the activation of YAP (7, 9, 10), and YAP is normally attenuated by the MST-LATS signaling network (10). The loss or reduced expression of LATS2 was reported in PCa and this was shown to be associated with hyperactivation of AR and upregulation of AR-dependent gene expression (38), with protein products known to promote PCa cell survival and inhibit apoptosis. In addition, mice lacking WW45 expression displayed hyperplasia in several organ sites (39). Here the inventors found that the induction of MST1 expression is sufficient to antagonize AR-driven gene expression and suppress PCa cell growth. Moreover, the inventors found that the growth suppressive effects of MST1 significantly declined in castration-resistant C4-2 cells in comparison to the effects of MST1 in castration-sensitive LNCaP parental cells, though both cell models expressed similar levels of MST1 protein. MST1 was identified as a negative regulatory component of PI3K-AKT signaling, and reduced MST1 expression was shown to correlate with PCa progression to the hormone-refractory metastatic state, which coincides with AKT activation (15). The inventors' data are consistent with this observation and indicate that the induction of MST1 expression sensitized C4-2 cells to growth suppression induced by PI3K inhibition. While not wishing to be bound by any one particular theory, these findings raise the possibility that deregulated-MST function may be associated with the emergence of the the hypothesis of whether one or both of these kinases have a direct role in prostate carcinogenesis and emergence of castration resistance.

PCa is the most commonly diagnosed cancer among men and the second leading cause of cancer death in Western countries (40, 41). Evidence indicates that cooperative AR and PI3K/AKT-mTOR pathway signaling is critical to human prostate tumor development and progression to the metastatic phenotype (42-44). Based on published studies (15, 16) and the inventors' present findings, the inventors propose a model (FIG. 7D) in which MST1/2 attenuates AR-dependent gene expression by interacting with the AR, which may lead to the alterations of the AR protein and/or transcriptional complexes, as well as by targeting upstream of the AR signal. An important implication from these observations is that deregulation of MST1 may account for the upregulation of AR and AKT signaling regulating cell survival. Therefore, disruption of the AR-AKT oncogenic network by MST1/2 alone and/or in combination with chemotherapeutic agents that target AR and PI3K/AKT-mTOR have important therapeutic implications.

### REFERENCES

1. Creasy C L, Chernoff J. Cloning and characterization of a member of the MST subfamily of Ste20-like kinases. Gene. 1995; 167:303-6.
2. Ling P, Lu T J, Yuan C J, Lai M D. Biosignaling of mammalian Ste20-related kinases. Cell Signal. 2008; 20:1237-47.
3. Ura S, Masuyama N, Graves J D, Gotoh Y. Caspase cleavage of MST1 promotes nuclear translocation and chromatin condensation. Proc Natl Acad Sci USA. 2001; 98:10148-53.
4. Teraishi F, Guo W, Zhang L, Dong F, Davis J J, Sasazuki T, et al. Activation of sterile20-like kinase 1 in proteasome inhibitor bortezomib-induced apoptosis in oncogenic K-ras-transformed cells. Cancer Res. 2006; 66:6072-9.
5. Oh S, Lee D, Kim T, Kim T S, Oh H J, Hwang C Y, et al. Crucial role for Mst1 and Mst2 kinases in early embryonic development of the mouse. Mol Cell Biol. 2009; 29:6309-20.
6. Wu S, Huang J, Dong J, Pan D. hippo encodes a Ste-20 family protein kinase that restricts cell proliferation and promotes apoptosis in conjunction with salvador and warts. Cell. 2003; 114:445-56.
7. Lu L, Li Y, Kim S M, Bossuyt W, Liu P, Qiu Q, et al. Hippo signaling is a potent in vivo growth and tumor suppressor pathway in the mammalian liver. Proc Natl Acad Sci USA. 107:1437-42.
8. Praskova M, Xia F, Avruch J. MOBKL1A/MOBKL1B phosphorylation by MST1 and MST2 inhibits cell proliferation. Curr Biol. 2008; 18:311-21.
9. Song H, Mak K K, Topol L, Yuri K, Hu J, Garrett L, et al. Mammalian Mst1 and Mst2 kinases play essential roles in organ size control and tumor suppression. Proc Natl Acad Sci USA. 107:1431-6.
10. Zhou D, Conrad C, Xia F, Park J S, Payer B, Yin Y, et al. Mst1 and Mst2 maintain hepatocyte quiescence and suppress hepatocellular carcinoma development through inactivation of the Yap1 oncogene. Cancer Cell. 2009; 16:425-38.
11. Harvey K F, Pfleger C M, Hariharan I K. The Drosophila Mst ortholog, hippo, restricts growth and cell proliferation and promotes apoptosis. Cell. 2003; 114:457-67.
12. Jia J, Zhang W, Wang B, Trinko R, Jiang J. The Drosophila Ste20 family kinase dMST functions as a tumor suppressor by restricting cell proliferation and promoting apoptosis. Genes Dev. 2003; 17:2514-9.
13. Udan R S, Kango-Singh M, Nolo R, Tao C, Halder G. Hippo promotes proliferation arrest and apoptosis in the Salvador/Warts pathway. Nat Cell Biol. 2003; 5:914-20.
14. Seidel C, Schagdarsurengin U, Blumke K, Wurl P, Pfeifer G P, Hauptmann S, et al. Frequent hypermethylation of MST1 and MST2 in soft tissue sarcoma. Mol Carcinog. 2007; 46:865-71.
15. Cinar B, Fang P K, Lutchman M, Di Vizio D, Adam R M, Pavlova N, et al. The pro-apoptotic kinase Mst1 and its caspase cleavage products are direct inhibitors of Akt1. EMBO J. 2007; 26:4523-34.
16. Cinar B, Mukhopadhyay N K, Meng G, Freeman M R. Phosphoinositide 3-kinaseindependent non-genomic signals transit from the androgen receptor to Akt1 in membrane raft microdomains. J Biol Chem. 2007; 282:29584-93.
17. Gioeli D, Black B E, Gordon V, Spencer A, Kesler C T, Eblen S T, et al. Stress kinase signaling regulates androgen receptor phosphorylation, transcription, and localization. Mol Endocrinol. 2006; 20:503-15.
18. Cinar B, Yeung F, Konaka H, Mayo M W, Freeman M R, Zhau H E, et al. Identification of a negative regulatory cis-element in the enhancer core region of the prostate-specific antigen promoter: implications for intersection of androgen receptor and nuclear factor-kappaB signalling in prostate cancer cells. Biochem J. 2004; 379:421-31.
19. Mukhopadhyay N K, Cinar B, Mukhopadhyay L, Lutchman M, Ferdinand A S, Kim J, et al. The zinc finger protein ras-responsive element binding protein-1 is a coregulator of the androgen receptor: implications for the role of the Ras pathway in enhancing androgenic signaling in prostate cancer. Mol Endocrinol. 2007; 21:2056-70.
20. Cinar B, Koeneman K S, Edlund M, Prins G S, Zhau H E, Chung L W. Androgen receptor mediates the reduced tumor growth, enhanced androgen responsiveness, and selected target gene transactivation in a human prostate cancer cell line. Cancer Res. 2001; 61:7310-7.
21. Cleutjens K B, van der Korput H A, van Eekelen C C, van Rooij H C, Faber P W, Trapman J. An androgen response element in a far upstream enhancer region is essential for high, androgen-regulated activity of the prostate-specific antigen promoter. Mol Endocrinol. 1997; 11:148-61.
22. Sun M, Yang L, Feldman R I, Sun X M, Bhalla K N, Jove R, et al. Activation of phosphatidylinositol 3-kinase/Akt pathway by androgen through interaction of p85alpha, androgen receptor, and Src. J Biol Chem. 2003; 278:42992-3000.
23. Wang Y, Kreisberg J I, Ghosh P M. Cross-talk between the androgen receptor and the phosphatidylinositol 3-kinase/Akt pathway in prostate cancer. Curr Cancer Drug Targets. 2007; 7:591-604.
24. Lin J, Adam R M, Santiestevan E, Freeman M R. The phosphatidylinositol 3′-kinase pathway is a dominant growth factor-activated cell survival pathway in LNCaP human prostate carcinoma cells. Cancer Res. 1999; 59:2891-7.
25. Pedram A, Razandi M, Sainson R C, Kim J K, Hughes C C, Levin E R. A conserved mechanism for steroid receptor translocation to the plasma membrane. J Biol Chem. 2007; 282:22278-88.
26. Xu K, Shimelis H, Linn D E, Jiang R, Yang X, Sun F, et al. Regulation of androgen receptor transcriptional activity and specificity by RNF6-induced ubiquitination. Cancer Cell. 2009; 15:270-82.
27. Fu M, Rao M, Wu K, Wang C, Zhang X, Hessien M, et al. The androgen receptor acetylation site regulates cAMP and AKT but not ERK-induced activity. J Biol Chem. 2004; 279:29436-49.
28. Nishida T, Yasuda H. PIAS1 and PIASxalpha function as SUMO-E3 ligases toward androgen receptor and repress androgen receptor-dependent transcription. J Biol Chem. 2002; 277:41311-7.
29. Gioeli D, Ficarro S B, Kwiek J J, Aaronson D, Hancock M, Catling A D, et al. Androgen receptor phosphorylation. Regulation and identification of the phosphorylation sites. J Biol Chem. 2002; 277:29304-14.
30. Guo Z, Dai B, Jiang T, Xu K, Xie Y, Kim O, et al. Regulation of androgen receptor activity by tyrosine phosphorylation. Cancer Cell. 2006; 10:309-19.
31. McCall P, Gemmell L K, Mukherjee R, Bartlett J M, Edwards J. Phosphorylation of the androgen receptor is associated with reduced survival in hormone-refractory prostate cancer patients. Br J. Cancer. 2008; 98:1094-101.
32. Ura S, Masuyama N, Graves J D, Gotoh Y. MST1-JNK promotes apoptosis via caspase-dependent and independent pathways. Genes Cells. 2001; 6:519-30.
33. Heemers H V, Tindall D J. Androgen receptor (AR) coregulators: a diversity of functions converging on and regulating the AR transcriptional complex. Endocr Rev. 2007; 28:778-808.
34. Hodgson M C, Astapova I, Cheng S, Lee L J, Verhoeven M C, Choi E, et al. The androgen receptor recruits nuclear receptor CoRepressor (N-CoR) in the presence of mifepristone via its N and C termini revealing a novel molecular mechanism for androgen receptor antagonists. J Biol Chem. 2005; 280:6511-9.
35. Liao G, Chen L Y, Zhang A, Godavarthy A, Xia F, Ghosh J C, et al. Regulation of androgen receptor activity by the nuclear receptor corepressor SMRT. J Biol Chem. 2003; 278:5052-61.
36. Guenther M G, Barak O, Lazar M A. The SMRT and N-CoR corepressors are activating cofactors for histone deacetylase 3. Mol Cell Biol. 2001; 21:6091-101.
37. Zeng Q, Hong W. The emerging role of the hippo pathway in cell contact inhibition, organ size control, and cancer development in mammals. Cancer Cell. 2008; 13:188-92.
38. Powzaniuk M, McElwee-Witmer S, Vogel R L, Hayami T, Rutledge S J, Chen F, et al. The LATS2/KPM tumor suppressor is a negative regulator of the androgen receptor. Mol Endocrinol. 2004; 18:2011-23.
39. Lee J H, Kim T S, Yang T H, Koo B K, Oh S P, Lee K P, et al. A crucial role of WW45 in developing epithelial tissues in the mouse. EMBO J. 2008; 27:1231-42.
40. Scher H I, Sawyers C L. Biology of progressive, castration-resistant prostate cancer: directed therapies targeting the androgen-receptor signaling axis. J Clin Oncol. 2005; 23:8253-61.
41. Mohler J L. Castration-recurrent prostate cancer is not androgen-independent. Adv Exp Med Biol. 2008; 617:223-34.
42. Edwards J, Krishna N S, Witton C J, Bartlett J M. Gene amplifications associated with the development of hormone-resistant prostate cancer. Clin Cancer Res. 2003; 9:5271-81.
43. Ghosh P M, Malik S N, Bedolla R G, Wang Y, Mikhailova M, Prihoda T J, et al. Signal transduction pathways in androgen-dependent and -independent prostate cancer cell proliferation. Endocr Relat Cancer. 2005; 12:119-34.
44. King J C, Xu J, Wongvipat J, Hieronymus H, Carver B S, Leung D H, et al. Cooperativity of TMPRSS2-ERG with PI3-kinase pathway activation in prostate oncogenesis. Nat Genet. 2009; 41:524-6.

What is claimed is:

1. A method of detecting cancer in a subject comprising:
obtaining a sample biopsy from a subject;
determining the expression level of MST1 and/or MST2 in the sample biopsy; and
comparing the expression level of MST1 and/or MST2 with the expression level of MST1 and/or MST2 from a control biopsy; wherein a lower level of expression of MST1 and/or MST2 in the sample biopsy, compared with the control biopsy, is indicative of cancer in the subject.

2. The method according to claim 1, wherein the cancer is prostate cancer.

3. The method according to claim 1, wherein the cancer is hormone-refractory metastatic cancer.

4. The method according to claim 1, wherein the subject is a human.

5. A method of prognosing cancer in a subject comprising:
obtaining a sample biopsy from a subject;
determining the expression level of MST1 and/or MST2 in the sample biopsy; and
comparing the expression level of MST1 and/or MST2 with the expression level of MST1 and/or MST2 from a control biopsy, wherein a lower level of expression of MST1 and/or MST2 in the sample biopsy, compared to the control biopsy, results in a poor prognosis of cancer in the subject.

6. The method according to claim 5, wherein the cancer is prostate cancer.

7. The method according to claim 5, wherein the cancer is hormone-refractory metastatic cancer.

8. The method according to claim 5, wherein the subject is human.

9. A method of suppressing cancer growth in a subject comprising directly and/or indirectly increasing the expression and/or presence of MST1 and/or MST2 in the subject.

10. The method according to claim 9, wherein the cancer is prostate cancer.

11. The method according to claim 9, wherein the cancer is hormone-refractory metastatic cancer.

12. The method according to claim 9, wherein the expression of MST1 and/or MST2 is increased in the tumor cells of the subject.

13. The method according to claim 9, wherein the subject is a human.

14. The method according to claim 9, further comprising administering a treatment selected from the group consisting of: brachytherapy, chemotherapy, cryosurgery, hormone therapy, radiation therapy, prostatectomy, and combinations thereof.

15. The method according to claim 14, wherein the hormone therapy comprises treatment selected from the group consisting of: suppressing a hormone, blocking a hormone and eliminating a hormone.

16. The method according to claim 15, wherein the hormone is an androgen.

17. The method according to claim 16, wherein the hormone is testosterone.

18. The method of claim 14, wherein the chemotherapy comprises administering a chemotherapeutic agent that targets AR and/or PI3K/AKT-mTOR.

19. A method of inhibiting cancer in a subject, comprising directly and/or indirectly increasing the expression and/or presence of MST1 and/or MST2 in the subject.

20. The method according to claim 19, wherein the cancer is prostate cancer.
